(19) United States
(12) Patent Application Publication
(10) Pub. No.: US 2022/0361940 A1
(43) Pub. Date: Nov. 17, 2022

(54) METHODS AND COMPOSITIONS FOR TREATING DERMATOLOGICAL CONDITIONS BEFORE, DURING, AND/OR AFTER ELECTROMAGNETIC RADIATION TREATMENT

(71) Applicant: AQUAVIT PHARMACEUTICALS, INC., New York, NY (US)

(72) Inventors: Sobin Chang, New York, NY (US); Jill Waibel, New York, NY (US)

(21) Appl. No.: 17/635,329

(22) PCT Filed: Aug. 10, 2020
(86) PCT No.: PCT/US20/45645

§ 371 Date: Feb. 14, 2022

Related U.S. Application Data
(60) Provisional application No. 62/885,898, filed on Aug. 13, 2019.

(51) Int. Cl. A61B 18/14; A61B 5/15; A61B 5/151; A61M 5/158

(52) U.S. Cl. CPC A61B 18/1477; A61B 5/150984; A61B 5/15136; A61M 5/158; A61B 2018/1425; A61M 2037/0023

(57) ABSTRACT

The present invention provides a method for treating a dermatological condition in a subject, comprising administering to the subjects skin a bioactive composition using a microneedle delivery device, wherein the bioactive composition comprises an effective amount of an anesthetic; administering an effective amount of electromagnetic radiation to the subjects skin to induce damage to the epidermis and/or dermis; and optionally administering to the subjects skin an effective amount of a composition to promote wound healing.

### FIELD OF THE INVENTION

The field of the invention relates generally to the field of medicine, specifically methods useful for treating dermatological conditions, in particular using sources of electromagnetic radiation.

### BACKGROUND OF THE INVENTION

There is an increasing demand for repair of or improvement to skin defects, which can be induced by aging, sun exposure, dermatological diseases, traumatic effects, and the like. Many treatments which use electromagnetic radiation have been used to improve skin defects by inducing a thermal injury to the skin, which results in a complex wound healing response of the skin. This leads to a biological repair of the injured skin.

In particular, the removal (or “skin peel”) of an outer layer of skin is used to treat conditions such as acne, age spots (superficial regions of excess melanin), shallow lesions (e.g. actinic keratoses), and aged skin.

The depth of the outer layer, or epidermis, varies, with ranges typically from 50-150 μm in thickness. The epidermis is separated from the underlying corium (dermis) by a germinative layer of columnar basal cells. The epidermal/dermal interface is characterized by undulations. The basal cells produce a continuing supply of keratinocytes, which are the microscopic components of the epidermis. Specialized cells called melanocytes, also reside in the basal cell layer and produce the pigment melanin. Although some of the melanin migrates toward the surface of the skin with the keratinocytes, the greatest concentration of melanin remains in the basal cell layer. The uppermost layer of the dermis, which is adjacent to the basal cell layer, is known as the papillary dermis, and the papillae range in width from 25-100 μm, separated by rete ridges (“valleys”) of comparable width.

Removal of the epidermis eliminates superficial sun damage, including keratoses, lentigenes, and fine wrinkling. Removal of the most superficial portions of the dermis, i.e. the uppermost papillary dermis, eliminates solar elastosis and ameliorates wrinkling, with little or no scarring.

Various treatments have been developed in recent years to induce thermal damage to the epidermis and/or dermis using electromagnetic radiation. One of the treatments is ablative laser skin resurfacing. A typical ablative laser skin resurfacing procedure comprises thermally damaging a region of the epidermis and a corresponding lower region of the dermis for promoting wound healing. Electromagnetic energy is directed towards a region of skin, thereby ablating the skin and removing both epidermal tissue and dermal tissue. This is considered to be an effective treatment protocol for photo aged or chronically aged skin, scars, superficial pigmented lesions, stretch marks, and/or superficial skin lesions.

One form of this treatment uses a short pulse carbon dioxide (CO₂) laser to coagulate a layer of skin to a depth of about 50-100 μm/pulse. This treatment is sometimes referred to as a “laser peel” or laser skin resurfacing. CO₂ laser radiation (in the 9-11 μm region of the infrared) is strongly absorbed by water (contained in all tissue). When the energy/unit volume absorbed by the tissue is sufficient to vaporize the water, a microscopically thin layer of tissue at the surface of the irradiated region is rendered necrotic. The skin is coagulated on the surface of the region irradiated by the infrared beam from the CO₂ laser. After irradiation, the dehydrated, necrotic surface layer can be mechanically removed, and additional irradiation can take place, this process being repeated until the desired depth of tissue is removed.

A newer treatment that is gaining in popularity uses a pulsed erbium YAG (Er:YAG) laser, emitting radiation at 2.94 μm in the infrared, where water absorption is even stronger than at CO₂ wavelengths. Er:YAG light is approximately 10 times more strongly absorbed in skin than CO₂ laser light. When compared to the effect of CO₂ laser irradiation, a shallower layer of skin absorbs the radiation and is vaporized and ablated from the surface, leaving a thinner thermally damaged and coagulated layer adjacent to the removed tissue. Damage has not been observed to exceed a depth of about 50 μm of collagen denaturation. See e.g., R. Kaufinann and R. Hibst, “Pulsed Erbium:YAG Laser Ablation in Cutaneous Surgery,” Lasers in Surgery and Medicine 19: 324-330(1996).

Treatment with the Er:YAG laser rejuvenates skin, with less pain, less inflammation, and more rapid healing than treatment with the CO₂ laser. The depth of penetration with the Er:YAG laser, being shallower, does not thermally stimulate new collagen growth as much as the CO₂ laser, so fine wrinkles are not eradicated as effectively. Dermatologists and cosmetic surgeons are, finding the Er:YAG laser preferable for younger patients who have superficial skin damage but less wrinkling, while the CO₂ laser is thought to be preferable for older patients who want to have fine wrinkles removed around the lips and the eyes. See e.g., Betsy Bates, “Dermatologists Give Er:YAG Laser Mixed Reviews,” Skin & Allergy News 28, No. 11: 42 (November 1997).

What is needed are new methods and compositions to alleviate the pain and discomfort associated with electromagnetic radiation treatments using lasers and which provide improved recovery and healing times.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

In one aspect, the invention provides a method for treating a dermatological condition in a subject, comprising

i) administering to the subject's skin a bioactive composition using a microneedle delivery device, wherein the bioactive composition comprises an effective amount of an anesthetic; and

ii) administering to the subject's skin an effective amount of electromagnetic radiation to induce damage to the epidermis and/or dermis.

In some embodiments, the method further comprises administering to the subject's skin an effective amount of a composition to promote wound healing. In some embodiments, the damage induced to the epidermis and/or dermis is thermal damage.

In some embodiments, the method comprises administering to the subject's skin an effective amount of a composition before, during and/or after an electromagnetic radiation treatment.

In some embodiments, the method of administration is coupled with an electromagnetic radiation that could be Intense Pulsed Light (IPL), Light Emitting Diode (LED), Titan and other infrared energy-based techniques and radio-frequency based procedures, such as Thermage.

In some embodiments, the wound healing composition comprises antimicrobial/antibiotic agents, such as silver or iodine, to either create a barrier to microorganisms or reduce microbial load. In some embodiments, these treatments are used more for managing the wound environment and moisture balance than actively promoting wound healing.

In some embodiments, the composition comprises one or more sacrificial proteolytic enzyme substrates and one or more antimicrobial agents. In some embodiments, the composition may contain preservatives such as sodium benzoate or chelators such as ethylenediaminetetraacetic acid (EDTA).

In some embodiments, the composition comprises diluents, adjuvants, excipients, vehicles, and other inert agents that act as carriers for other bioactive agents. In some embodiments, the composition comprises one or more substrates including, but not limited to, collagen, gelatin, elastin, casein, albumin, fibrinogen, fibronectin, and combinations and hydrolysates thereof. In certain embodiments, proteins for use as sacrificial substrates are hydrolyzed or partially hydrolyzed by treatment with a strong acid or base.

In some embodiments, the composition comprises one or more antimicrobial agents including, but not limited to, components of aloe vera, ashitaba, bacteriophage, beta-defensin, quaternary ammonium compound, chlorhexidine, copper, dispersin B, essential oil, gentamicin, lactoferrin, lysostaphin, N-halamines, nitric oxide, oleic acid, PLU C, polyhexanide biguanide (PHMB), bacteriocin, selenium, silver compound, triclosan, zinc, and combinations thereof.

In some embodiments, the composition comprises bioresorbable materials that includes, but are not limited to, polydioxanone, polyhydroxybutyrate, polyhydrozyvalerate, polyaminoacids polyorthoesters, polyvinly alcohol, chitosan, oxidized regenerated cellulose, hyaluronic acid, alginate, collagen, a modified collagen, such as gelatin or derivatives of any of the above.

In some embodiments, the composition comprises extracellular matrix proteins such as fibrin, collagen or fibronectin, and synthetic or naturally occurring polymers, including bioabsorbable or non-absorbable polymers, such as polylactic acid (PLA), polyglycolic acid (PGA), polylactide-co-glycolide (PLGA), polyvinylpyrrolidone, polycaprolactone, polycarbonates, polyfumarates, caprolactones, polyamides, polysaccharides (including alginates (e.g., calcium alginate) and chitosan), hyaluronic acid, polyhydroxybutyrate, polyhydroxyvalerate, polydioxanone, polyorthoesthers, polyethylene glycols, poloxamers, polyphosphazenes, polyanhydrides, polyamino acids, polyacetals, polycyanoacrylates, polyurethanes (e.g., GranuFoam®), polyacrylates, ethylene-vinyl acetate polymers and other acyl substituted cellulose acetates and derivatives thereof, polystyrenes, polyvinyl chloride, polyvinyl fluoride, poly(vinylimidazole), chlorosulphonated polyolefins, polyethylene oxide, polyvinyl alcohol, Teflon®, and nylon.

In some embodiments, the aforementioned compositions may be heated or melted before administering using the microneedle delivery device.

In another aspect, the invention provides a microneedle delivery device comprising an anesthetic composition for use in the claimed methods.

In another aspect, the invention provides a microneedle delivery device comprising a wound healing composition for use in the claimed methods.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is a view of a handheld microneedle injection apparatus. The syringe ejection volume is automatically controlled and dispenses into an interchangeable head containing one or several needles. The diagram shows the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 2 is an image of a screw on a microneedle head.

FIG. 3 is a schematic representation of a device in a syringe configuration. Alternative configurations include vial- and capsule-loaded configurations. The device holds a syringe (2) for automatic injection via one or more microneedles in the microneedle head. Ejection volume is controlled by an information processor (9). Other elements are noted: the motor or actuator (4) to control the piston (3), exchangeable and controllable needle head (1) and cam system and dial to adjust needle injection depth (5), and needle head ejector (10). Information is shown to the user in a display panel that may include a manual or touchscreen control panel (12) and data is stored in a storage unit (11) that may be removable. The needle head (1) may be controlled by an actuator (13).

FIG. 4 provides three additional views of a microneedle device. Microneedle components: (A) microneedles, (B) housing of the needles and (C) a reservoir.

FIG. 5 is a diagram showing the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 6 provides a depiction of the utility feature conferred by the circular or flat O-Rings. Said features enable enhanced liquid handling capabilities as evidenced by an airtight mechanism which facilitates the efficient and uniform delivery of treatment solutions to the skin. Said features are positioned at the interface of the cap and the reservoir channel so as to effectively prevent the leakage of treatment solution dosages. The RFID chip+O-ring depiction has been expanded. The cap/cover (1) will interface with the vial or container (5) containing a certain compound (6). The connection of both the cap/cover and the container may be sealed with a threaded opening (2). While pressure is applied vertically through the twisting motion of the thread, the rubber O-ring (3) seals the two interfaces (1) and (5) together. A ratchet mechanism (4) at the end will lock the cap in place. Embedded inside the rubber O-ring is a RFID chip (7) which material is shock, pH, temperature, and ozone resistant. The RFID chip will be stable enough under different environments to be able to effectively transmit data for applications such as data security, quality assurance/control, and logistics (8).

FIG. 7A-7B depict a utility feature conferred by the circular or flat O-Rings (FIG. 7A). Said features enable obvious and non-obvious advantages conferred by excellent weather and ozone resistance, temperature resistance (FIG. 7B) and the resistance to pH induced degradation of the butyl rubber or halogenated butyl rubber in comparison to other industrial rubbers and further addresses the stability of the material in the context of medical device utility, end user performance and pharmacological agent turbidity. Said features effectively enable enhanced material durability while preventing the leakage and inefficient delivery of treatment solution dosages with time.

FIG. 8 illustrates anti-unlock safety features of an O ring in a microneedle device.

FIG. 9 illustrates anti-unlock safety features of an O ring in a microneedle device.

FIG. 10 illustrates anti-unlock safety features of an O ring in a microneedle device.

FIG. 11 illustrates anti-unlock safety features of an O ring in a microneedle device.

FIG. 12 illustrates an exemplary microneedle drug delivery device.

FIG. 13A and FIG. 13B illustrate a co-packaging design of a kit with an exemplary microneedle drug delivery device along with a vial containing lidocaine. The kit contents to include but not limited to, three varying components (1. Bioactive compounds pre-packaged such as lidocaine, 2. microchannel delivery device and 3. standard syringe; as a complete set in any size and shape box, and packaged in any order, L to R, top to bottom, layered, circular, etc.

FIG. 14 provides an exemplary microneedle device for treating dermatological conditions before, during and/or after electromagnetic radiation treatment.

FIG. 15 provides internal assembly of parts of the device of FIG. 14.

FIG. 16 provides a view of the assembled internal parts of FIG. 14.

FIG. 17 provides a view of the assembled internal parts of FIG. 14.

FIG. 18 provides an external push assembly view of the device of FIG. 14.

FIG. 19 provides a view of the device of FIG. 14.

FIG. 20 provides a view of the device of FIG. 14.

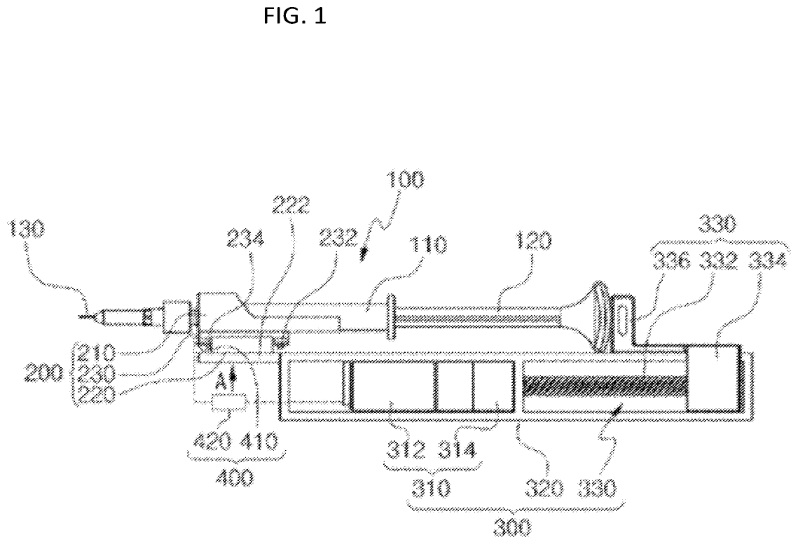

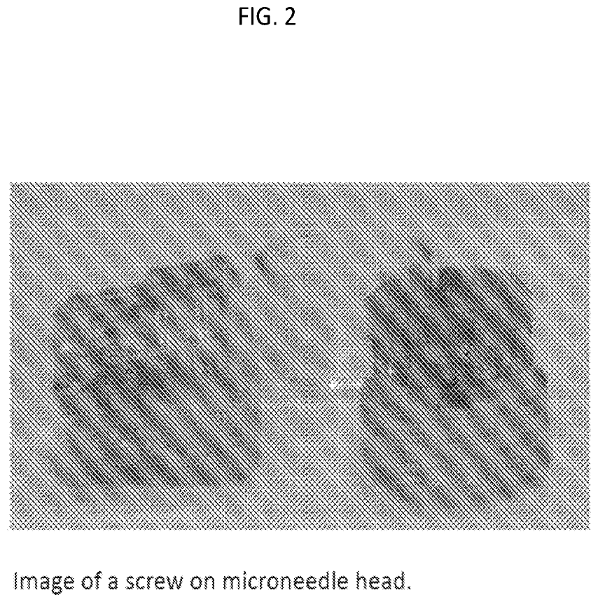

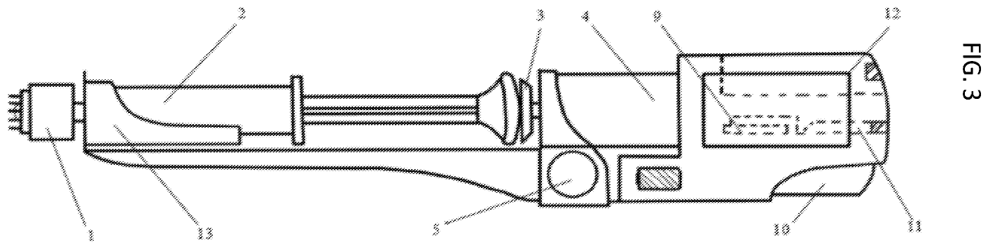

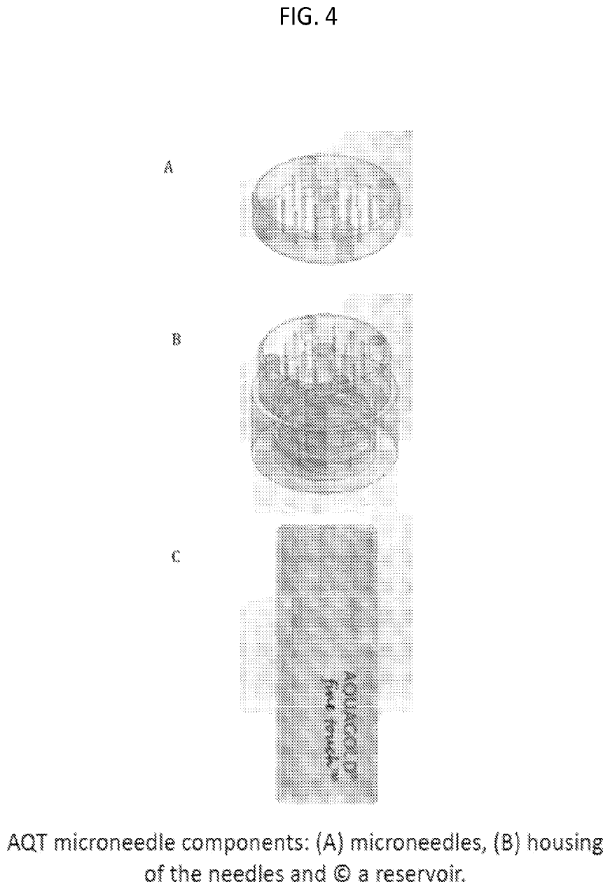

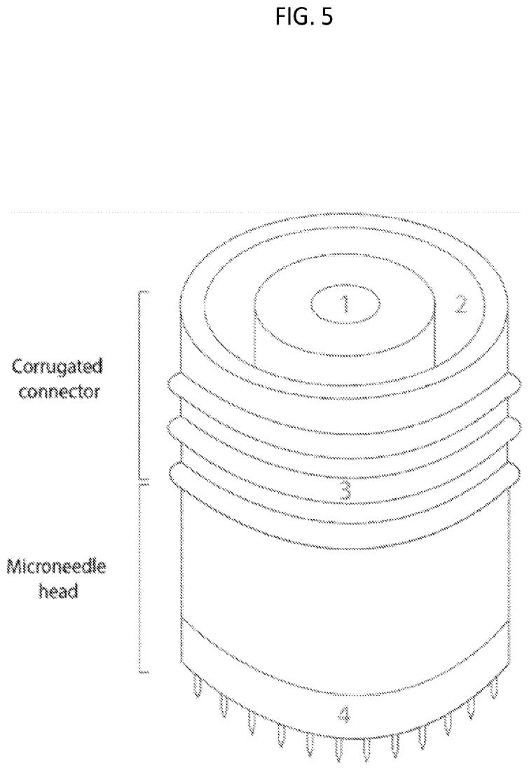

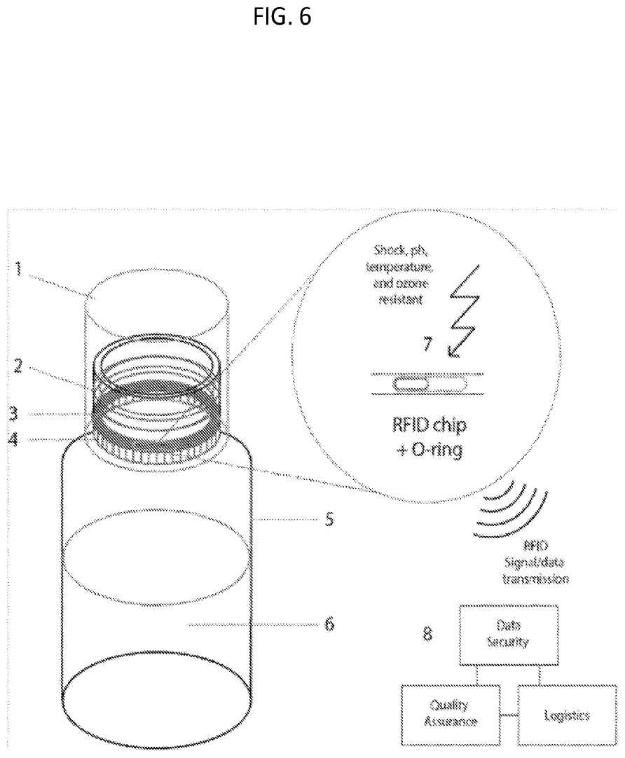

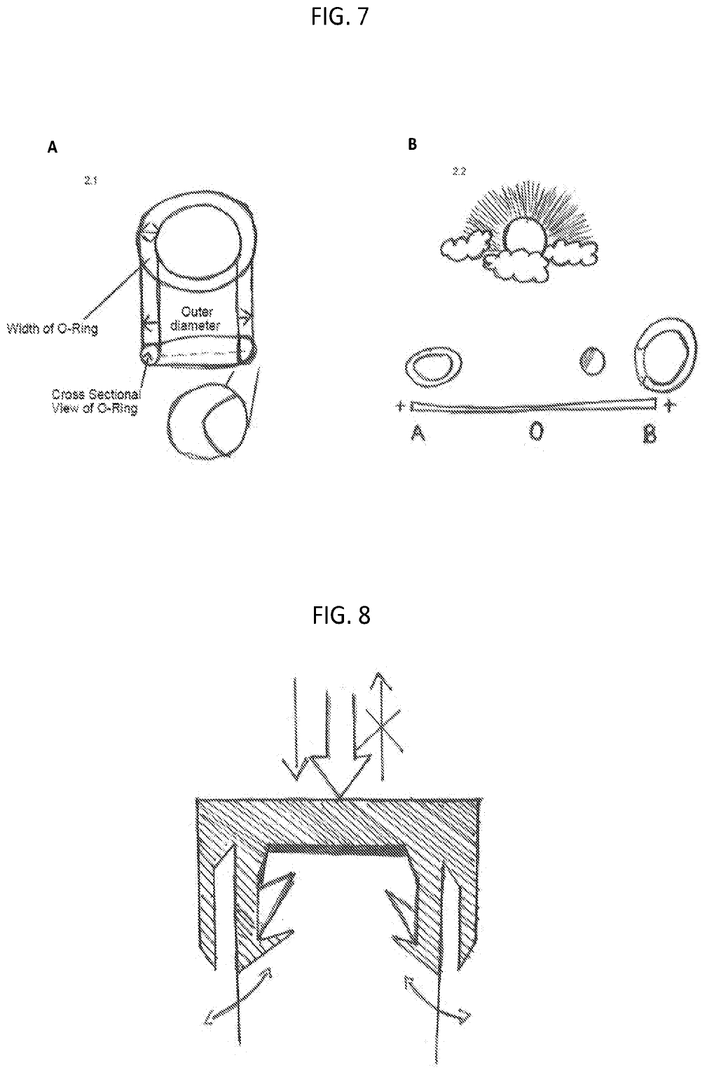

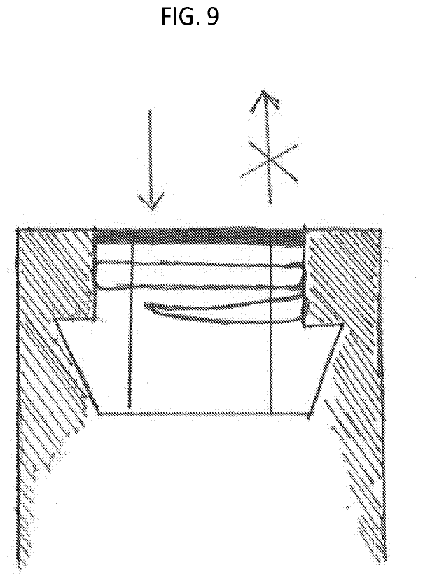

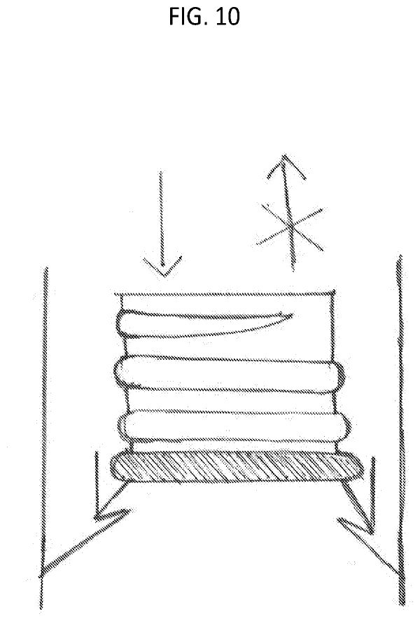

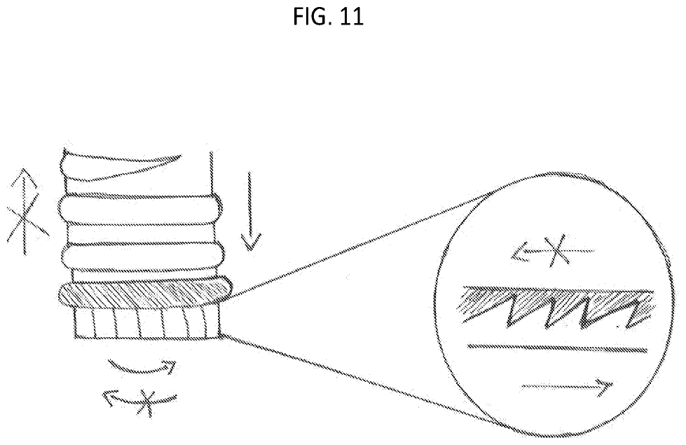

### DETAILED DESCRIPTION OF THE INVENTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

In one embodiment, the invention provides a method for treating a dermatological condition in a subject, comprising

i) administering to the subject's skin a bioactive composition using a microneedle delivery device, wherein the bioactive composition comprises an effective amount of an anesthetic; and

ii) administering an effective amount of electromagnetic radiation to the subject's skin to induce damage to the epidermis and/or dermis.

In some embodiments, the method further comprises administering to the subject's skin an effective amount of a composition to promote wound healing.

In some embodiments, the source of electromagnetic radiation is a laser.

In some embodiments, the damage induced to the epidermis and/or dermis is thermal damage.

The term “subject” as used herein is not limiting and is used interchangeably with patient. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like.

The dermatological condition can include, for example, photo aged skin, chronically aged skin, scarring, acne, hair removal, stretch marks, lesions, sun damage, keratoses, lentigenes, sagging skin, wrinkles and combinations thereof.

In some embodiments, the electromagnetic radiation is administered by a non-natural source. In some embodiments, the electromagnetic radiation is administered using a laser source. In some embodiments, the amount administered induces damage to the epidermis and/or dermis. In some embodiments, the damage is thermal damage. In some embodiments, the electromagnetic radiation removes one or more layers of the subject's skin. In some embodiments, the method produces localized tissue damage generally sparing the surrounding tissues.

In some embodiments, the laser source is selected from the group consisting of an argon ion gas laser, a carbon dioxide (CO₂) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof.

In some embodiments, the laser source is an Er:YAG laser. In some embodiments, the laser source is a carbon dioxide (CO₂) gas laser.

In some embodiments, the laser source is PDL, IPL/BBL, Alexandrite, Fraxel/Thulium, Ruby, Diode, KTP, 1440 nm, Radiofrequency, Ultrasound, Coolsculpting, Nd:YAG laser.

In some embodiments, the wavelength of the electromagnetic radiation used in the treatment can be between about 200 nm and about 20,000 nm. The wavelength of the electromagnetic radiation can be selected based on the absorption strength of various components within the tissue and the scattering strength of the tissue. The wavelength of the electromagnetic radiation can be chosen to target a particular chromophore, such as, for example, water, elastin, collagen, sebum, hemoglobin, myoglobin, melanin, keratin, or other endogenous or exogenous molecules present in the tissue. Wavelengths that are primarily absorbed by water present in the tissue, such as, for example, 1550 nm, can be used. The wavelength of the electromagnetic radiation treatment can be within the near infrared spectrum, such as, for example, between about 700 nm and about 1400 nm. Wavelengths in the visible spectrum, such as, for example, between about 400 nm and about 700 nm are also useful. Ultraviolet electromagnetic radiation within the range of between about 200 nm to about 400 nm can also be used.

In some embodiments, the electromagnetic radiation can have a wavelength that is highly absorbed in water. Cellular water absorbs electromagnetic radiation and transforms the electromagnetic radiation into heat. Wavelengths larger than 190 nm, such as wavelengths in the range from 190 nm to 10600 nm, from 700 nm to 1600 nm, and about 1550 nm can used. The source of electromagnetic radiation used to provide the treatment can be capable of providing one wavelength or a range of wavelengths or can be tunable across a range of wavelengths. One or more sources of electromagnetic radiation can be used to produce a variety of different wavelengths or wavelength ranges used in the dermatological treatment. In some embodiments, the electromagnetic radiation source can be adapted to selectively produce pulses of electromagnetic radiation at a frequency of between about 0 and about 50,000 pulses per second, or between about 0 and about 1,000 pulses per second. In one example, an electromagnetic radiation source can emit a beam having pulse energy per treatment spot of between about 1 mJ and about 1000 mJ, or between about 10 mJ and about 30 mJ, with each pulse having a pulse duration per treatment spot between about 0.1 ms and about 30 ms, or about 1 ms.

In some embodiments, the dermatological treatment can be used, for example, to produce non-ablative coagulation of an epidermal and/or a dermal layer of tissue. Typically, for this purpose, an optical fluence incident to a tissue area greater than about 5 J/cm², such as an optical fluence in the range from about 10 J/cm² to about 1000 J/cm², is adequate for coagulating tissue. Generally, the optical fluence is adapted to the wavelength and the tissue to be treated. If various dermatological effects are desired, a treatment device can be selected with the capacity to produce source parameters suitable for other types of tissue treatment. For example, if ablation of an epidermal layer of the tissue is desired, a treatment device can be used with the capability to emit a beam of electromagnetic radiation with a wavelength of about 2940 nm and optical fluence higher than about 10 J/cm².

In some embodiments, the dermatological condition to be treated is acne. In some embodiments, the chromophore in the tissue that is targeted includes hemoglobin and/or water. In some embodiments, the wavelength of the electromagnetic radiation administered is about 1450-1540 nm.

In some embodiments, the dermatological condition to be treated is scarring. In some embodiments for fresh scars, hemoglobin is the abundant chromophore and fibroblasts are the targets. In some embodiments for treating scars, lasers with a 590 nm cut off filter can be used. In some embodiments, Er:Glass 1540 nm fractional lasers can be used.

In some embodiments, the dermatological condition to be treated are pigmented lesions and/or tattoos. Melanin absorbs light at a wide range of wavelengths, e.g., from 250 nm to 1200 nm and therefore almost any laser with sufficient power causing thermal denaturation can be used to remove benign pigmented lesions of the epidermis. In dermal pigmented lesions the target chromophore is intracellular pigment melanosomes or tattoo particles. In some embodiments, continuous wave lasers like CO₂ (10,600 nm) or Er-YAG (2940 nm) with water as a target chromophore in epidermis can be used for removing the superficial pigmented skin, especially seborrheic keratosis. In some pigmented lesions, the melanosomes and melanocytes are clustered so they can act as a larger body of chromophore. In these lesions, melanin specific wavelengths can lead to lesion clearance.

In some embodiments, the administration of electromagnetic radiation is an ablative resurfacing treatment. In some embodiments, the ablative resurfacing treatment is for promoting tissue tightening for wrinkles and rhytides, skin lightening for superficial sun damage dyschromia, lentigines or actinic keratosis, or skin leveling for atrophic acne or chicken pox scars. In some embodiments, the ablative resurfacing can be performed with CO₂ laser (10,600 nm), with either continuous wave or ultra-short pulse mode, with or without a scanner. In some embodiments, the ablative resurfacing can be performed with an Er:YAG (2940 nm) laser. In some embodiments, a combined erbium and CO₂ laser can be used.

In some embodiments, the administration of electromagnetic radiation is a non ablative laser resurfacing treatment. In some embodiments, the devices which can be used target the vasculature or water in the dermis. In some embodiments, the source includes a pulsed dye lasers (PDL) or intense pulsed light (IPL) device. In another embodiment, the source includes mid infra-red lasers that target water in the dermis to effect dermal heat deposition. In some embodiments, the source can include 1320 nm Nd:YAG, 1450 nm Diode and 1540 nm Erbium:glass lasers.

In some embodiments, a non-ablative resurfacing treatment can be used to treat fine lines. In some embodiments, vascular elements can be targeted with a PDL or an IPL with a 590 nm filter. In some embodiments, a pigment disorder can be treated with an IPL with 645 nm filter or near infra-red laser of 1064 nm Nd:YAG. In some embodiments, the subject is administered electromagnetic radiation with a wavelength of about 1320, about 1450 or about 1540 nm.

In some embodiments, the administration of electromagnetic radiation is a fractional resurfacing treatment. Fractional resurfacing is an ablative resurfacing of the skin but only on a small fraction of the skin. In some embodiments, the area treated with each pass is not more than 10% of the total surface area. In some embodiments, the wavelength used is 1540 nm with an Erbium:glass laser with either a scanning device (FRAXEL) or an array of micro lenses (Matisse™).

In some embodiments of fractional resurfacing treatment, depending on the desired size and depth of the treatment zones, the wavelength of the electromagnetic radiation used can be selected from the group consisting of between about 1100 nm and about 2500 nm, between about 1280 nm and about 1350 nm, between about 1400 nm and about 1500 nm, between about 1500 nm and about 1620 nm, between about 1780 nm and 2000 nm, and combinations thereof. Wavelengths longer than 1500 nm and wavelengths with absorption coefficients in water of between about 1 cm⁻¹ and about 30 cm⁻¹ can be used if the goal is to get deep penetration with small treatment zones. The shorter wavelengths generally have higher scattering coefficients than the longer wavelengths.

The composition that can be administered in accordance with the methods is not limiting. In some embodiments, a composition comprising one or more bioactive agents is administered before, during and/or after electromagnetic radiation treatment. In some embodiments, one or more bioactive agents is elected from the group consisting of one or more vitamins, one or more minerals, retinol, retinoic acid, a bleaching/whitening agent, collagen, a neuromodulator, poly-L-lactic acid, an anesthetic and combinations thereof. In some embodiments, the neuromodulator comprises botulinum toxin (e.g., botulinum toxin of serotype A, B, C, D, E, F or G).

In some embodiments, one or more bioactive agents that can be added is discussed below.

The microneedle delivery device can be used to deliver a bioactive composition comprising an effective amount of an anesthetic. In some embodiments, a microneedle delivery device can also be used to administer to the subject's skin one or more compositions to promote wound healing. In some embodiments, the bioactive composition that comprises the anesthetic also includes one or more components to promote wound healing. In some embodiments, the subject is administered one or more wound healing compositions after receiving the electromagnetic radiation treatment.

The bioactive composition comprising an effective amount of an anesthetic is administered prior to administering the electromagnetic radiation. In some embodiments, the bioactive composition comprising an effective amount of an anesthetic is administered about 1 hour prior to the electromagnetic radiation treatment, about 30 minutes prior to the electromagnetic radiation treatment, or about 15 minutes prior to the electromagnetic radiation treatment. In some embodiments, the bioactive composition comprising an effective amount of an anesthetic is administered immediately prior to the electromagnetic radiation treatment.

In some embodiments, the anesthetic is selected from the group consisting of mepivacaine, articaine, bupivacaine, ropivacaine, prilocaine, chloroprocaine, lidocaine, tetracaine and combinations thereof. In some embodiments, the bioactive composition comprising the anesthetic further comprises one or more of epinephrine or levonordefrin.

In some embodiments, the bioactive composition is a tumescent anesthetic saline solution. In some embodiments, the composition comprises about 0.05-2% anesthetic such as lidocaine, and epinephrine or levonordefrin in an amount of about 1:100,000 to about 1:10,000,000. In some embodiments, the tumescent anesthetic solution comprises about 1% lidocaine and about 1:1,00,000 epinephrine. In some embodiments, the anesthetic solution comprises one or more buffers. In some embodiments, the buffer is sodium bicarbonate.

Vitamins, or vital nutrients, and minerals are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium. When referring to a vitamin, it would be understood that all chemical forms of the vitamin are contemplated.

B vitamins are a group of water-soluble vitamins that play important roles in cell metabolism. The B vitamins are B1 (thiamine), B2 (riboflavin), B3 (niacin), B5 (pantothenic acid), B6 (pyridoxine), B7 (biotin), B12 (cobalamin) and folic acid. The B vitamins play an important role in many aspects of the body's functioning, and a vitamin B deficiency can have a serious impact on overall health.

Vitamin B supplements are known in the art: such formulations are limited in terms of absorption (oral dosage forms) or may require a hospital visit (IV therapy) at significant cost in terms of time and expense.

Collagen is a type of fibrous protein found most often in the skin, flesh, and connective tissue of vertebrates. In mammals, it is the most abundant protein in the body, and provides structural support for major tissues and organs. In the skin, it is responsible for providing structure, firmness, and smoothness, and it is often a decrease in collagen production that leads to chronic aging. For this reason, collagen is often injected or topically introduced to the skin in attempts to slow or reverse the effects of aging.

Vitamins and minerals, or vital nutrients, are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium.

Vitamin B12, also called cobalamin, is a water-soluble vitamin with a key role in the normal functioning of the brain and nervous system, and for the formation of blood. It is one of the eight B vitamins. It may be involved in the metabolism of every cell of the human body, especially affecting DNA synthesis and regulation, but also fatty acid synthesis and energy production. Vitamin B12 may also be involved in maintenance of the central nervous system and has been used to affect memory loss, Alzheimer's disease, boosting mood, energy and concentration, boost the immune system, and slow aging. Vitamin B12 may also play a role in heart disease, lowering high homocysteine levels (which may contribute to heart disease), male infertility, diabetes, sleep disorders, depression, mental disorders, weak bones (osteoporosis), swollen tendons, AIDS, inflammatory bowel disease, asthma, allergies, a skin disease called vitiligo, preventing cervical and other cancers, and skin infections. Two common forms of Vitamin B12 are cyanocobalamin and methylcobalamin.

Vitamin B12 deficiency may cause macrocytic anemia, fatigue, loss of appetite, loss of balance, weakness, and mood disturbances. It also may cause serious neurologic and neuropsychiatric illness such as paresthesias, ataxia, and memory loss. Vitamin B12 absorption may be impaired at the level of the stomach, where intrinsic factor is produced, or at the level of the terminal ileum, where intrinsic factor bound to vitamin B12 is absorbed.

Niacin and nicotinamide, also known as niacinamide, are forms of vitamin B3. Nicotinamide is the amide of nicotinic acid (vitamin B3/niacin). Nicotinamide is a water-soluble vitamin and is part of the vitamin B group. Nicotinamide may be used for preventing vitamin B3 deficiency and related conditions such as pellagra. Each of these forms of vitamin B3 may be used for schizophrenia, hallucinations due to drugs, Alzheimer's disease and age-related loss of thinking skills, chronic brain syndrome, depression, motion sickness, alcohol dependence, and fluid collection (edema).

Vitamin B1, also known as thiamine, is a water-soluble vitamin and may be utilized for metabolizing carbohydrates and production of energy. Vitamin B1 also may aid in the function of the heart and cardiovascular system and the nervous system.

Vitamin B6, also known as pyridoxine, may be involved in many aspects of macronutrient metabolism, neurotransmitter synthesis, histamine synthesis, hemoglobin synthesis and function and gene expression. Vitamin B6 may assist with cellular metabolism, supports the immune system, with formation of red blood cells and maintenance of healthy brain function. Vitamin B6 may be used for Alzheimer's disease, attention deficit-hyperactivity disorder (ADHD), Down syndrome, autism, diabetes and related nerve pain, sickle cell anemia, migraine headaches, asthma, carpal tunnel syndrome, night leg cramps, muscle cramps, arthritis, allergies, acne and various other skin conditions, and infertility. It is also may be used to treat dizziness, motion sickness, preventing the eye disease age-related macular degeneration (AMD), seizures, convulsions due to fever, and movement disorders (tardive dyskinesia, hyperkinesis, chorea), as well as for increasing appetite and helping people remember dreams. Vitamin B6 may be used for acne, leprosy, attention deficit-hyperactivity disorder (ADHD), memory loss, arthritis, preventing premenstrual headache, improving digestion, protecting against toxins and pollutants, reducing the effects of aging, lowering blood pressure, improving circulation, promoting relaxation, improving orgasm, and preventing cataracts. Vitamin B6 deficiency may cause anemia due to insufficient production of hemoglobin.

Vitamin B2, also known as riboflavin, releases energy from carbohydrates and may be used for preventing low levels of riboflavin (riboflavin deficiency), cervical cancer, and migraine headaches. It also may be used for treating riboflavin deficiency, acne, muscle cramps, burning feet syndrome, carpal tunnel syndrome, and blood disorders such as congenital methemoglobinemia and red blood cell aplasia. It also may be used for increasing energy levels; boosting immune system function; maintaining healthy hair, skin, mucous membranes, and nails; slowing aging; boosting athletic performance; promoting healthy reproductive function; canker sores; memory loss, including Alzheimer's disease; ulcers; burns; alcoholism; liver disease; sickle cell anemia; and treating lactic acidosis brought on by treatment with a class of AIDS medications called NRTI drugs.

The term “vitamin B6” encompasses multiple forms of vitamin B6 suitable for human administration. Several forms of the vitamin are known, but pyridoxal phosphate (PLP; “pyridoxine”) is the active form and may be used as a cofactor in many reactions of amino acid metabolism, including transamination, deamination, and decarboxylation. Pyridoxine may be used in enzymatic reactions affecting the release of glucose from glycogen.

Vitamin C, also known as ascorbic acid, is an antioxidant. Vitamin C may be used to protect against free radicals and promote a healthy immune system, wound healing, and forming healthy skin. More specifically, ascorbic acid may be used to prevent and treat scurvy, a disease caused by a lack of vitamin C in the body. People with high intakes of vitamin C from fruits and vegetables may have a lower risk of getting many types of cancer, such as lung, breast, and colon cancer.

Vitamin B5, also known as pantothenic acid, has skincare benefits. For example, it increases the degree of hydration of the skin, reduces the trans-epidermal water loss and keeps the elasticity and smoothness of the skin. Vitamin B5 may be used in acne treatments and may be used to reduce itchiness of the skin.

Zinc is an essential mineral found in cells throughout the body. Zinc is required for protein synthesis and collagen formation, and may be used to promote a healthy immune system and assist in wound healing. It may also be used for muscular growth and contraction and to protect the liver from chemical damage such as which can occur with anesthetics or other drugs or toxins. Zinc may also be utilized in bone formation. Zinc deficiency may contribute to fatigue, susceptibility to infection, and slow wound healing.

Ahseutasanchin is a unique pigment belonging to the carotenoid family. It exhibits antioxidant capacity against free radicals.

Vitamin E can boost the immune system and protect people against toxins such as air pollution, neurological disease such as Alzheimer's disease, and diabetes. As an antioxidant, Vitamin E can remove free radicals that damage the cell structure. Owing to this property, another well-known health benefit for Vitamin E is in skin and hair care.

Vitamin D helps intestines absorb nutrients and is essential for calcium utilization, ensuring strong bones and robust immune system.

Selenium displays antioxidant properties that regenerate vitamin C and vitamin E, thereby decreasing the aging of the skin and protecting cells from damage. Moreover, selenium also benefits the immune system and protects our body against various infections.

Glutathione(GSH) is an antioxidant which prevents damage to important cellular components caused by free radicals. The strong antioxidant effect of glutathione helps keep cells running smoothly and it also helps the liver remove chemicals that are foreign to the body, such as drugs and pollutants.

Anthocyanidins have a wide range of biological activities including antioxidant, anti-inflammatory, antimicrobial and anti-cancer activities. In addition they display a variety of effects on blood vessels, platelets and lipoproteins able to reduce the risk of coronary heart diseases.

EPA is a form of omega-3 fatty acids which can reduce cellular inflammation.

DHA is a building block of tissue in the brain and retina of the eye. It helps with forming neural transmitters, such as phosphatidylserine, which is important for brain function. EPA and DHA are also well-known for improving skin conditions. Their anti-inflammatory properties help prevent various skin ailments. EPA and DHA can also reduce the damage caused by overexposure to the sun and negative impacts of UV rays.

Lecithin acts as a solvent for cholesterol, triglycerides, and other fats. Therefore, it helps to prevent such ailments as high blood pressure, stroke, heart disease, hardening of the arteries, etc. Also, lecithin plays a vital role in the absorption of nutrients out of the blood stream into the cells.

CoQ10 helps to combat fatigue, boosts immune system, fight against free radicals, and keep cells both inside the body and in the skin healthy. The CoQ10 level decreases as people get older, resulting in an impeded ability to produce collagen and elastin, and the loss of collagen and elastin causes our skin wrinkle and sag.

Magnesium can benefit blood pressure and help prevent sudden cardiac arrest and stroke. It also plays a role in detoxification processes and therefore is important for helping to prevent damage from environmental chemicals, heavy metals and other toxins.

Vitamins and minerals, or vital nutrients, are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium.

Bleaching/whitening agents that may be used in the compositions described herein include, but are not limited to, hydroquinone, kojic acid, ascorbic acid, magnesium ascorbyl phosphate or ascorbyl glucosamine, hydroquinone, licorice extract (e.g., Glycyrrhiza Glabra (licorice) root extract), an alpha MSH antagonist (e.g. undecylenoyl phenylalanine), phytic acid, monobenzyl ether of hydroquinone, azelaic acid, kojic acid, mequinol, retinoids (e.g., tretinoin, adapalene), soy proteins, alpha-hydroxy acids (e.g., glycolic acid), trichloroacetic acid, salicylic acid, hydroquinone-beta-D-glucopyranoside, paper mulberry, glabridin, 4-isopropylcetchol, aleosin, N-acetyl-4-S-cycteaminylphenol, N-propionyl-4-S-cysteaminylphenol, N-acetyl glucosamine, tranexaminc acid and mixtures thereof.

Retinols used in the compositions described herein include, but are not limited to, retinoic acid.

Collagen is a type of fibrous protein found most often in the skin, flesh, and connective tissue of vertebrates. In mammals, it is the most abundant protein in the body, and provides structural support for major tissues and organs. In the skin, it is responsible for providing structure, firmness, and smoothness, and it is often a decrease in collagen production that leads to chronic aging. For this reason, collagen is often injected or topically introduced to the skin in attempts to slow or reverse the effects of aging (Varani J, Dame M K, Rittie L, Fligiel S E G, Kang S, Fisher G J, Voorhees J J: Decreased Collagen Production in Chronically Aged Skin. Am J Pathol. 2006 June; 168(6):1861-1868. PubMed PMCID: PMC1606623).

Hyaluronic acid (HA) is involved in cartilage resilience and skin repair, has been applied medically for decades for a number of different uses including, for example, cartilage resilience and skin repair. Among the most common of these medical applications employ injectable delivery, for example to treat joint pain, or topical delivery, for example to treat dermatitis. Cosmetically, it is often used as an active agent in facial filler injections to smooth wrinkles and in topical creams and gels to rejuvenate the skin and combat the aging process. Hyaluronic acid includes both cross-linked and non-cross-linked hyaluronic acids.

Botulinum toxin, a neurotoxic protein, is used cosmetically and therapeutically for treatment of facial lines and wrinkles, upper motor neuron syndrome, excessive sweating, cervical dystonia, chronic migraine, and overactive bladder. The toxin is generally injected into the subcutaneous muscles at the target areas, and works by temporarily (for a period of six weeks to eight months, depending on the location and the dose) inhibiting the release of acetylcholine at the neuromuscular junction and thus paralyzing the muscles achieve the desired affects (BOTOX (onabotulinumtoxinA) [prescribing information]. Irvine, Calif. Allergan, Inc. January 2013). Botulinum toxin refers to any botulinum toxin, including but not limited to botulinum toxin type A, botulinum toxin type B, botulinum toxin type Cl, botulinum toxin type D, botulinum toxin type E, botulinum toxin type F and botulinum toxin type G. Botulinum toxin type A includes, for example, Botox, Dysport and Xeomin. Botulinum toxin type B includes, for example, MyoBloc. Botulinum toxin may be provided in a liquid or powder form. A powdered form may be, for example, a sterile, lyophilized preparation. Lyophilized preparations may be reconstituted prior to application. Alternatively, botulinum toxin may be provided as a sterilized pre-dissolved solution. Botulinum toxin may be formulated in an amount of about 0.01 to about 60 units.

“MicroBotox” or “Purtox” as used herein, refers to instances when diluted Botox is injected in multiple very small doses in a treated area. The effects of the Botox are more evenly spread over the areas treated and the risks of having areas over-treated is reduced. Use of MicroBotox generally results in a more natural look (i.e., less frozen) and the dosage of Botox administered is reduced. For some patients suffering from recalcitrant acne problems, MicroBotox (referred to as “mesoBotox” when used in this situation) can be injected very superficially into the facial skin. Following dilution, microBotox may be formulated in an amount of 0.1 to about 99% of the compositions. For example, one would use 0.1 to about 100 units of onabotulinum toxin diluted with at least 2.5 cc of saline.

Minoxidil is a vasodilator that was originally administered orally as a treatment for hypertension, but was found to have the additional effect of slowing hair loss and promoting hair growth. It is now a common topical treatment for androgenic hair loss, and is thought to achieve hair regrowth by increasing the blood flow (and thus the availability of oxygen and vital nutrients) to the hair follicles, stimulating them to resume normal functioning (Olsen E A, Whiting D, Bergfeld W, Miller J, Hordinsky M, Wanser R, Zhang P, Kohut B: A multicenter, randomized, placebo-controlled, double-blind clinical trial of a novel formulation of 5% minoxidil topical foam versus placebo in the treatment of androgenetic alopecia in men. J Am Acad Dermatol. 2007 Aug. 29. PubMed PMID: 17761356).

Platelet-rich plasma (PRP) is blood plasma that has been enriched by platelets, and is prepared by separating whole blood via centrifugation and then collecting the plasma-rich layers that emerge. Because it has five times the baseline platelet concentration of plasma (.about.100,000 platelets per microliter as opposed to the baseline of .about.20,000 platelets per microliter), it contains a number of different growth factors (proteins that stimulate tissue growth, the release of which can be induced by the addition of thrombin and calcium chloride. PRP injections have been used clinically for several years as a treatment for nerve, bone, and muscle injuries, and have been used cosmetically to reverse damage to the skin and to promote dermal strength and rejuvenation (Borrione P, Gianfrancesco A D, Pereira M T, Pigozzi F: Platelet-rich plasma in muscle healing. Am J Phys Med Rehabil. 2010 October; 89(10):854-61. PubMed PMID: 20855985).

Poly-L-lactic Acid (PLLA) is a type of dermal filler used in the treatment of facial lipoatrophy (the gradual loss of facial fat, generally due to aging). PLLA, upon entering the skin, provides immediate structural support to the skin and also promotes the neo-synthesis of collagen, hiding sunken areas. Over time, it is converted by the body into harmless lactic acid, gradually transferring the load to the recently synthesized collagen (SCULPTRA Aesthetic (injectable poly-L-lactic acid) [prescribing information]. Bridgewater, N.J. Sanofi-Aventis U.S. LLC. May 2012).

Bimatoprost is a prostaglandin prodrug that is administered topically to control the progression of Glaucoma and to treat ocular hypertension. Since 2008, the application of this drug has evolved to encompass a cosmetic formulation for the lengthening and darkening of eyelashes and is thought to confer an improved appearance by delivering bimatoprost—a growth stimulating analog—circambient to the hair follicles at the edge of the eyelid.

In one embodiment, the composition comprises a therapeutically effective amount of one or more of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, (HA), collagen, botulinum toxin (e.g., botulinum toxin of serotype A, B, C, D, E, F or G), platelet-rich plasma (PRP), poly-L-lactic acid (PLLA), and optionally lidocaine with epinephrine, a chemical stabilizer and optionally a preservative.

In some embodiments, the wound healing composition comprises an effective amount of growth factors, platelet rich plasma, cells, engineered cells, stem cells, zinc, a collagen byproduct, a collagen precursor, hyaluronic acid, a vitamin, an antioxidant, an amino acid, a supplemental mineral, C-E-Ferulic (ferulic acid and pure vitamin C and E), poly-L-lactic acid, hyaluronic acid filler, corticosteroid (e.g., triamcinolone), 5-fluorouracil, latisse or combinations thereof.

In some embodiments, the wound healing composition comprises antimicrobial/antibiotic agents, such as silver or iodine, to either create a barrier to microorganisms or reduce microbial load. In some embodiments, these treatments are used more for managing the wound environment and moisture balance than actively promoting wound healing.

In some embodiments, the composition comprises one or more sacrificial proteolytic enzyme substrates and one or more antimicrobial agents. In some embodiments, the composition may contain preservatives such as sodium benzoate or chelators such as ethylenediaminetetraacetic acid (EDTA).

In some embodiments, the composition comprises diluents, adjuvants, excipients, vehicles, and other inert agents that act as carriers for other bioactive agents. In some embodiments, the composition comprises one or more substrates including, but not limited to, collagen, gelatin, elastin, casein, albumin, fibrinogen, fibronectin, and combinations and hydrolysates thereof. In certain embodiments, proteins for use as sacrificial substrates are hydrolyzed or partially hydrolyzed by treatment with a strong acid or base.

In some embodiments, the composition comprises one or more antimicrobial agents including, but not limited to, components of aloe vera, ashitaba, bacteriophage, beta-defensin, quaternary ammonium compound, chlorhexidine, copper, dispersin B, essential oil, gentamicin, lactoferrin, lysostaphin, N-halamines, nitric oxide, oleic acid, PLU C, polyhexanide biguanide (PHMB), bacteriocin, selenium, silver compound, triclosan, zinc, and combinations thereof.

In some embodiments, the composition comprises bioresorbable materials that includes, but are not limited to, polydioxanone, polyhydroxybutyrate, polyhydrozyvalerate, polyaminoacids polyorthoesters, polyvinly alcohol, chitosan, oxidized regenerated cellulose, hyaluronic acid, alginate, collagen, a modified collagen, such as gelatin or derivatives of any of the above.

In some embodiments, the composition comprises extracellular matrix proteins such as fibrin, collagen or fibronectin, and synthetic or naturally occurring polymers, including bioabsorbable or non-absorbable polymers, such as polylactic acid (PLA), polyglycolic acid (PGA), polylactide-co-glycolide (PLGA), polyvinylpyrrolidone, polycaprolactone, polycarbonates, polyfumarates, caprolactones, polyamides, polysaccharides (including alginates (e.g., calcium alginate) and chitosan), hyaluronic acid, polyhydroxybutyrate, polyhydroxyvalerate, polydioxanone, polyorthoesthers, polyethylene glycols, poloxamers, polyphosphazenes, polyanhydrides, polyamino acids, polyacetals, polycyanoacrylates, polyurethanes (e.g., GranuFoam®), polyacrylates, ethylene-vinyl acetate polymers and other acyl substituted cellulose acetates and derivatives thereof, polystyrenes, polyvinyl chloride, polyvinyl fluoride, poly(vinylimidazole), chlorosulphonated polyolefins, polyethylene oxide, polyvinyl alcohol, Teflon®, and nylon.

In some embodiments, the aforementioned compositions may be heated or melted before administering using the microneedle delivery device.

In some embodiments, the compositions, methods and kits herein can employ a microneedle device to administer the compositions. A microneedle array can be used to deliver a drug directly to the dermis (the second layer of skin). In some embodiments, the microneedle arrays or needleless injector devices as disclosed herein deliver the bioactive agent or drug into the dermal and epidermal junction area. In another embodiment, the microneedle device does not penetrate into the dermal layer but only disrupts the superficial portion of the skin, referred to as stratum corneum.

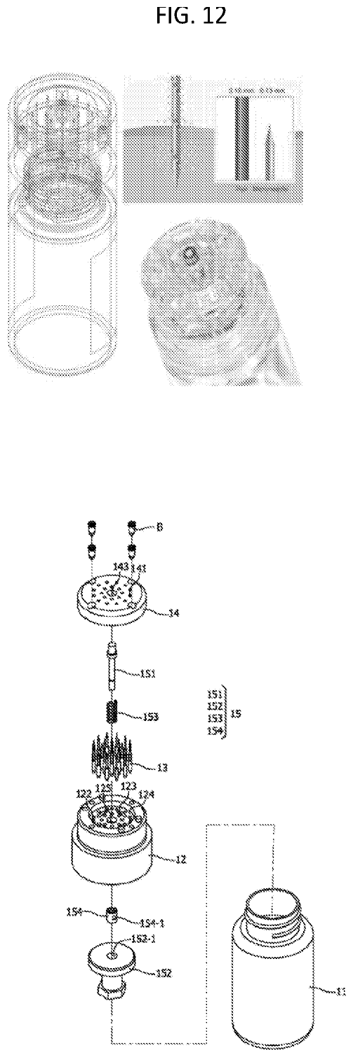

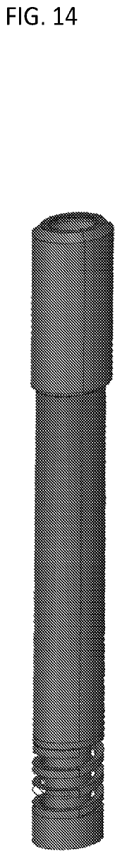

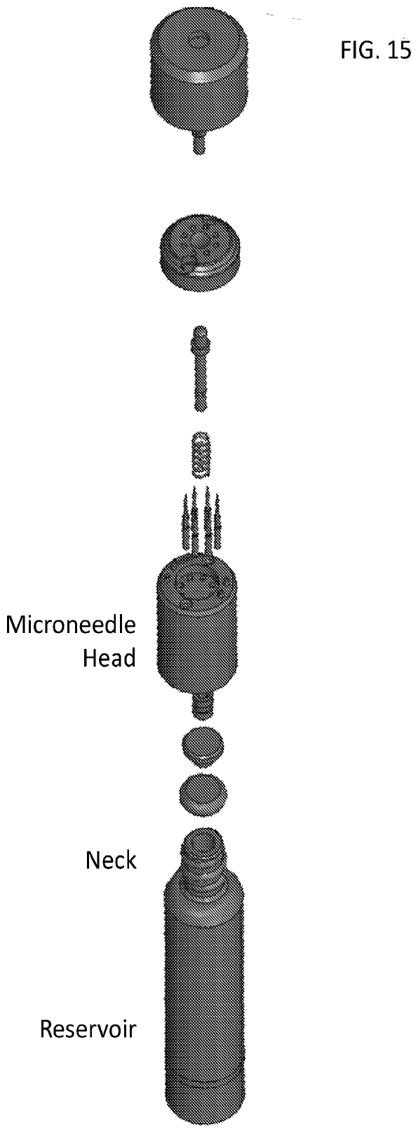

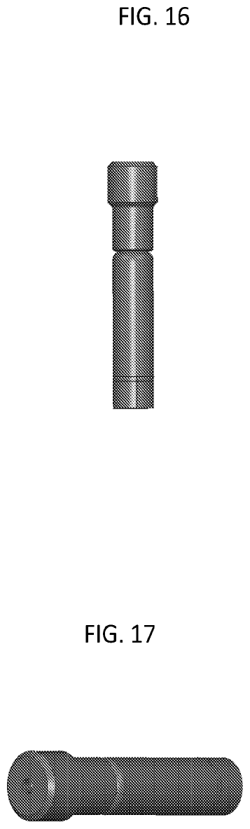

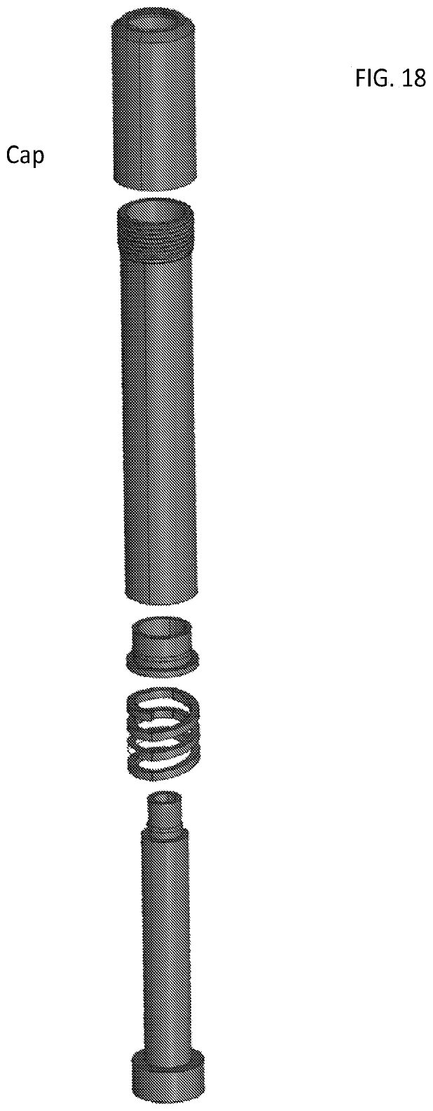

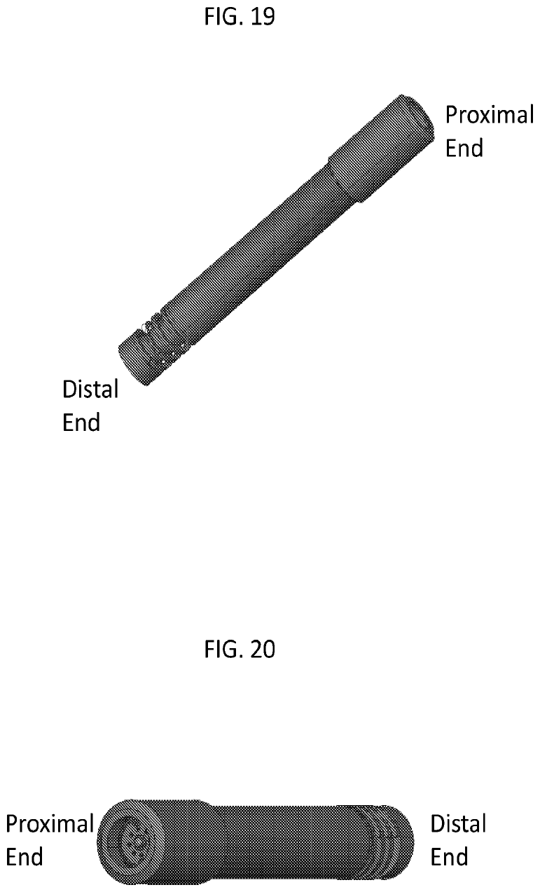

In some embodiments, the microneedle delivery device useful in the methods of the invention is depicted in FIG. 12. In some embodiments, the microneedle drug delivery device is as described in Korean Patent No. 10-1582822, which is incorporated by reference herein in its entirety. In some embodiments, the microneedle device useful in the methods of the invention is depicted in FIGS. 14-20.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the bioactive composition contained in the reservoir into the skin.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the plurality of microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles.

In some embodiments, the microneedle delivery device is repeatedly pressed against the subject's skin to deliver the composition to the area of the skin to be treated. In some embodiments, the microneedle delivery device is repeatedly pressed about 10, about 20, about 30, about 40, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 or more times to administer the composition.

In some embodiments, the composition is administered by the microneedle delivery device with a repeated motion of penetrating the microneedle delivery device into the skin of the subject. In some embodiments, the composition is delivered into the skin by passing through the one or multiple grooves along the outer wall of the microneedle. In some embodiments, the microneedles are non-hollow.

In some embodiments, the microneedle delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microneedle delivery device is capable of delivering compositions directly to the epidermal, dermal and subcuticular layers of the skin. Therefore, it should be understood that further embodiments developed for use with non-hollow or hollow microneedle systems of delivery by those skilled in the art fall within the spirit and scope of this disclosure.

In another aspect, a microneedle device for use in the methods described herein is a device such as described in U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a sealing mechanism that is secured to the tips of the microneedles. In another instance, the device may further include means for providing feedback to indicate that delivery of the material from the reservoir has been initiated or completed. An osmotic pump may be included to expel the material from the reservoir. One or more microneedles may be disposed at an angle other than perpendicular to the substrate. In certain instances, the at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising the composition into the skin in a process that is painless compared to injectables. Administration can result in easy and precise delivery of a composition comprising the composition with generally no bruising, pain, swelling and bleeding caused by the injection.

The device may include means, manual or mechanical, for compressing the reservoir, creating a vacuum, or otherwise using gravity or pressure to drive the composition from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microneedle device enhances transportation of drugs across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons.

The array may be attached to a reservoir to hold the substances to be delivered, and this reservoir may be any volume (about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

In one embodiment, the direct application device comprises a single or an array of microneedles that will serve not only as an anchor to the skin, but also as a collagen stimulator platform (via collagen induction therapy) to accelerate skin healing or combat age-related decreases in collagen neosynthesis. Each microneedle will have one or multiple grooves inset along its outer wall. This structural feature of the dermal delivery device allows liquids stored in a reservoir at the base of each needle to travel along the needle shaft into the tissue. Altogether, this innovation is a functional treatment regimen applicator that enables the optimal restorative efficacy of bioactive formulations delivered beneath the surface of the skin.

A microneedle array can consist of from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles can each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array will be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The delivered substances may be of varying viscosities and concentration, from 0.01% to 100%, and will be administered via the microneedle array either independently or in conjunction with the aforementioned compositions.

The reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

A cadre of microneedles housed in a plastic or polymer composite head can be used to deliver treatment solutions, directly to the dermis, the second layer of skin or the topical layer of skin. The application of a mechanical load to the pin of the spring lock system enables the micro needles to puncture the epidermal barrier and deliver the desired substances directly to the dermis for faster, more efficient, and more effective absorption by the skin. The Spring Plate mechanism, housed in the plastic or polymer composite cartridge, is effectively the interface whereby the manual direct application mechanism calibrates the controlled delivery of the treatment solution into the skin.

Provided herein is a system, comprising one or more bioactive formulations and a microneedle delivery system, wherein at least one bioactive formulation comprises one or more anesthetics. In some embodiments, the system comprises at least one or more bioactive formulations to promote wound healing.

Other suitable microneedle devices such as pens, rollers, and patches, may be used to delivery or collect liquids when interfaced with tissue. They can be manually or electronically applied to achieve a stamping motion, or rolled over the skin. Such devices also include, in some instances, an autoinjector.

Provided herein is a system, comprising a bioactive formation and a microneedle delivery system, wherein the bioactive formulation comprises one or more anesthetics, such as lidocaine.

In another embodiment, provided herein is a system comprising a microneedle delivery device and a bioactive formulation, comprising (a) a single or an array of microneedles with a channel for liquid form to pass through; (b) a reservoir chamber with bioactive compounds or formulations customized or prefilled; (c) a plunger that releases the said compounds or formulations; (d) an optional security material to anti-reverse lock the microneedles and the chamber; (e) an optional adapter to fit any reservoir chamber; (f) an optional RFID smart label connecting to artificial intelligence portal; (g) an optional cloud-based informatics platform connected to system (big data predictive analytics); (h) an optional UV blocking agent for the chamber; and (i) an optional addition of a patch form using the groove in the microneedles for the purpose of sustained and/or extended release of the compositions, formulations or microchips.

The optional security material may comprise a Butyl Rubber O-Ring for optimizing the end-user utility and or experience. In one embodiment, the optimized butyl rubber O-ring confers pivotal advantages for the delivery of therapeutic treatment solutions to a tissue bed. In another embodiment, the optimized butyl rubber O-ring enable the overall improvement in performance and or utility of the underlying medical device. In another embodiment, the optimized butyl rubber O-ring is a flat or circular Butyl Rubber O-Ring. In another embodiment, the optimized butyl rubber O-ring serves as a liquid handling and leakage prevention seal, and improves the system efficiency characterized by an airtight mechanism for applying uniform volumes of treatment solutions. In another embodiment, the optimized butyl rubber O-ring further substantiates the overall suitability of the material in the context of medical device performance.

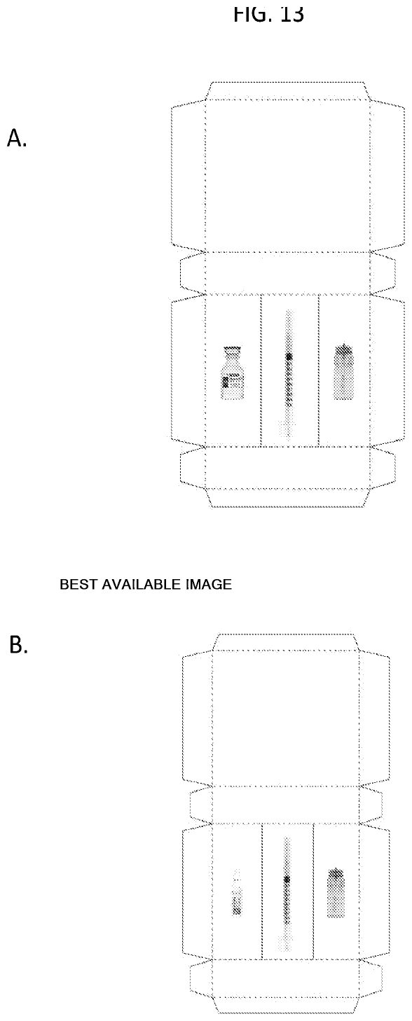

In another aspect, the invention provides a kit for use in the methods herein, comprising one or more compositions and microneedle devices as described herein, optionally comprising instructions for use and optionally further comprising one or more aqueous solvents for dissolving any of the bioactive components. In some embodiments, the kit is as shown in FIG. 13 and includes a vial comprising an anesthetic agents such as lidocaine, a microneedle delivery device, and optionally a hypodermic needle for injecting the bioactive composition into the microneedle delivery device. In some embodiments, the solvent is a buffered solution. In some embodiments, the solution is saline solution. In some embodiments, the solvent is water. In some embodiments, the kit comprises one or more anesthetics. In some embodiments, the kit further comprises one or more containers or packets comprising one or more bioactive agents in solid or liquid form. In some embodiments, the one or more bioactive agents is selected from any of the bioactive agents herein, and can include, e.g., one or more vitamins, one or more minerals, retinol, retinoic acid, a bleaching/whitening agent, collagen, a neuromodulator, poly-L-lactic acid, an anesthetic and combinations thereof.

In another embodiment, the invention provides a microneedle delivery device for use in the claimed methods comprising a bioactive composition comprising an effective amount of an anesthetic.

In another embodiment, the invention provides a microneedle delivery device for use in the claimed methods comprising an effective amount of a wound healing composition.

The amount of the therapeutic agents of the invention which will be effective in promoting anesthesia and wound healing can be determined by standard clinical techniques. The precise dose to be employed in the formulation will also depend on the judgment of the practitioner and each subject's circumstances. Effective doses can be extrapolated from dose-response curves derived from in vitro or animal model test systems.

One skilled in the art can readily determine an appropriate dosage regimen for administering therapeutically active agents of the invention to a given subject. For example, the compound(s) or composition(s) can be administered to the subject in one administration or multiple administrations. Where a dosage regimen comprises multiple administrations, it is understood that the effective amount of the compound(s) or composition(s) administered to the subject can comprise the total amount of the compound(s) or composition(s) administered over the entire dosage regimen. The exact amount will depend on the purpose of the treatment, the subject to be treated, and will be ascertainable by a person skilled in the art using known methods and techniques for determining effective doses. In some embodiments, the amount of the therapeutic agent that can be administered includes between about 0.1 μg/kg to about 100 mg/kg. In some embodiments, the amount of the therapeutic agent that can be administered includes between about 1.0 μg/kg to about 10 mg/kg.

In some embodiments, the compositions are formulated in accordance with routine procedures as a pharmaceutical composition adapted for intravenous, subcutaneous or parenteral administration to human beings. Typically, compositions for administration are solutions in sterile isotonic aqueous buffer. Where necessary, the composition can also include a solubilizing agent. Generally, the ingredients are supplied either separately or mixed together in unit dosage form, for example, as a dry lyophilized powder or water free concentrate in a hermetically sealed container such as an ampoule indicating the quantity of active agent. Where the compositions are administered by injection, an ampoule of sterile water for injection or saline can be provided so that the ingredients can be mixed prior to administration.

The therapeutic agents can also be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In certain embodiments, the compositions are pharmaceutical compositions. In some embodiments, formulations are prepared for storage and use by combining the active agents with a pharmaceutically acceptable vehicle (e.g. carrier, excipient) (Remington, The Science and Practice of Pharmacy 20th Edition Mack Publishing, 2000). In some embodiments, pharmaceutical compositions of the present invention are characterized as being at least sterile and pyrogen-free. As used herein, “pharmaceutical formulations” include formulations for human and veterinary use. Pharmaceutical compositions of the invention can be packaged for use in liquid form, or can be lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

For a broad overview of controlled delivery systems, for example, of polypeptides, see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules can contain the therapeutically active agents as a central core. In microspheres the therapeutic can be dispersed throughout the particle. Particles, microspheres, and microcapsules smaller than about 1 μm are generally referred to as nanoparticles, nanospheres, and nanocapsules, respectively. Microparticles are typically around 100 μm in diameter. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

In some embodiments, polymers can be used for controlled release of compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes can be used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### EXAMPLES

### Example 1. Anesthetic Drug Delivery Using a Microneedle Drug Delivery Device in Non-Ablative Laser Resurfacing Treatment

Three patients undergoing nonablative laser treatment were randomized and treated in a split-faced fashion. Half of the face underwent application of traditional numbing cream (bupivacaine 0.1%, lidocaine 10%, tetracaine 8%). The other half of the face underwent application of local lidocaine 1% with epinephrine using a microneedle device of the invention described herein (AQUAGOLD applicator). Following an hour of numbing to allow for the topical cream to take effect, the side with topical cream was wiped down and patients were treated with a laser combination of PDL/IPL/Thulium with customized settings based on individual treatment needs. Following treatment, all three patients reported no significant differences in sensation between the two sides regardless of analgesia route, supporting the effectiveness of microneedle delivery of anesthetics in laser treatment. Injectable lidocaine also does not require more than a few minutes to take effect versus the long wait times associated with topical numbing cream.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method for treating a dermatological condition in a subject, comprising
i) administering to the subject's skin a bioactive composition using a microneedle delivery device, wherein the bioactive composition comprises an effective amount of an anesthetic; and
ii) administering an effective amount of electromagnetic radiation to the subject's skin to induce damage to the epidermis and/or dermis.

2. The method of claim 1, wherein the electromagnetic radiation is administered using a laser source.

3. The method of any of claims 1-2, wherein the laser source is a carbon dioxide laser.

4. The method of any of claims 1-2, wherein the laser source is a Er:YAG laser.

5. The method of any of claims 1-4, wherein the electromagnetic radiation removes one or more layers of the subject's skin.

6. The method of any one of claims 1-5, wherein the microneedle delivery device comprises
i) a plurality of microneedles, wherein the microneedles are hollow or non-hollow, wherein one or multiple grooves are inset along an outer wall of the microneedles; and
ii) a reservoir that holds the bioactive composition to be delivered, wherein the reservoir is attached to or contains a means to encourage flow of the bioactive composition contained in the reservoir into the skin;
wherein the administering comprises a repeated motion of penetrating the microneedle delivery device into the skin of the subject,
wherein the bioactive composition is delivered into the skin by passing through the one or multiple grooves along the outer wall of the microneedle.

7. The method of claim 6, wherein the microneedles are non-hollow.

8. The method of any of claims 6-7, wherein the means to encourage flow of the bioactive composition contained in the reservoir into the skin is selected from the group consisting of a plunger, pump and suction mechanism.

9. The method of any of claims 6-7, wherein the means to encourage flow of the bioactive composition contained in the reservoir into the skin is a mechanical spring loaded pump system.

10. The method of any of claims 1-9, wherein the microneedles have a single groove inset along the outer wall of the microneedle, wherein the single groove has a screw thread shape going clockwise or counterclockwise around the microneedle.

11. The method of any of claims 1-10, wherein the microneedles are from 0.1 mm to about 2.5 mm in length and from 0.01 mm to about 0.5 mm in diameter.

12. The method of any of claims 1-11, wherein the microneedles are composed of gold.

13. The method of any of claims 6-12, wherein the plurality of microneedles comprises an array of microneedles in the shape of a circle.

14. The method of any of claims 1-13, wherein the microneedles are made of 24-carat gold plated stainless steel and comprise an array of 20 microneedles.

15. The method of any of claims 1-14, wherein the anesthetic is selected from the group consisting of mepivacaine, articaine, bupivacaine, ropivacaine, prilocaine, chloroprocaine, lidocaine, tetracaine and combinations thereof.

16. The method of any of claims 1-15, wherein the bioactive compositing comprising the anesthetic further comprises one or more of epinephrine or levonordefrin.

17. The method of any of claims 1-16, wherein the bioactive composition comprises epinephrine and lidocaine.

18. The method of claim 17, wherein the bioactive composition is a tumescent anesthetic saline solution comprising about 0.05-2% lidocaine, and epinephrine in an amount of about 1:100,000 to about 1:10,000,000.

19. The method of claim 18, wherein the tumescent anesthetic solution comprises about 1% lidocaine and 1:1,00,000 epinephrine.

20. The method of any of claims 1-19, wherein the anesthetic solution comprises one or more buffers.

21. The method of claim 20, wherein the buffer is sodium bicarbonate.

22. The method of any of claims 1-21, wherein the method further comprises administering a composition to promote wound healing by a microneedle delivery device.

23. The method of claim 22, wherein the microneedle delivery device comprises
i) a plurality of microneedles, wherein the microneedles are hollow or non-hollow, wherein one or multiple grooves are inset along an outer wall of the microneedles; and
ii) a reservoir that holds the wound healing composition to be delivered, wherein the reservoir is attached to or contains a means to encourage flow of the wound healing composition contained in the reservoir into the skin;
wherein the administering comprises a repeated motion of penetrating the microneedle delivery device into the skin of the subject,
wherein the wound healing composition is delivered into the skin by passing through the one or multiple grooves along the outer wall of the microneedle.

24. The method of claim 23, wherein the microneedles are non-hollow.

25. The method of any of claims 23-24, wherein the means to encourage flow of the wound healing composition contained in the reservoir into the skin is selected from the group consisting of a plunger, pump and suction mechanism.

26. The method of any of claims 23-24, wherein the means to encourage flow of the wound healing composition contained in the reservoir into the skin is a mechanical spring loaded pump system.

27. The method of any of claims 22-26, wherein the microneedles have a single groove inset along the outer wall of the microneedle, wherein the single groove has a screw thread shape going clockwise or counterclockwise around the microneedle.

28. The method of any of claims 22-27, wherein the microneedles are from 0.1 mm to about 2.5 mm in length and from 0.01 mm to about 0.5 mm in diameter.

29. The method of any of claims 22-28, wherein the microneedles are composed of gold.

30. The method of any of claims 22-29, wherein the plurality of microneedles comprises an array of microneedles in the shape of a circle.

31. The method of any of claims 22-30, wherein the microneedles are made of 24-carat gold plated stainless steel and comprise an array of 20 microneedles.

32. The method of any of claims 1-31, wherein the wound healing composition comprises an effective amount of growth factors, platelet rich plasma, cells, engineered cells, stem cells, zinc, a collagen byproduct, a collagen precursor, hyaluronic acid, a vitamin, an antioxidant, an amino acid, a supplemental mineral, C-E-Ferulic (ferulic acid and pure vitamin C and E), poly-L-lactic acid, hyaluronic acid filler, corticosteroid (e.g., triamcinolone), 5-fluorouracil, latisse or combinations thereof.
